(19) United States
(12) Patent Application Publication
(10) Pub. No.: US 2004/0076597 A1
(43) Pub. Date: Apr. 22, 2004

(54) Substances for inducing or intensifying the tanning mechanisms of the skin and cosmetic or dermatological formulations comprising same

(75) Inventors: Werner Berens, Thuine (DE); Franz Stab, Echem (DE); Rainer Wolber, Hamburg (DE); Thomas Blatt, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/373,341

(22) Filed: Feb. 24, 2003

Related U.S. Application Data
(63) Continuation of application No. PCT/EP01/09658, filed on Aug. 21, 2001.

(30) Foreign Application Priority Data
Aug. 24, 2000 (DE) 100 41 482.6

(51) Int. Cl. A61K 7/06; A61K 7/11

(52) U.S. Cl. 424/70.13

(57) ABSTRACT

The invention is a cosmetic or dermatological formulation comprising one or more substances selected from the group of AGEs and precursors thereof. The present invention also includes methods of using cosmetic or dermatological formulations comprising AGEs and precursors thereof, for example, in inducing or intensifying the tanning the skin.

### CROSS-REFERENCE TO RELATED APPLICATIONS

This is a continuation application of PCT/EP01/09658, filed Aug. 21, 2001, which is incorporated herein by reference in its entirety, and also claims the benefit of German Priority Application No.100 41 482.6, filed Aug. 24, 2000.

### FIELD OF THE INVENTION

The present invention relates to substances for inducing and intensifying the tanning mechanisms of the skin and cosmetic or dermatological formulations comprising same.

### BACKGROUND OF THE INVENTION

Artificial tanning is understood as meaning all measures which impart a tanned appearance to the skin without the action of solar rays. Various possibilities are in principle conceivable for this purpose, such as, for example,

physical coloring by applying make-up,

coloring by chemical reactions with the help of self-tanning preparations,

tanning by (oral) intake of carotenoids, and

artificial irradiation using technically produced light sources.

In this connection, it must be taken into consideration that, in the case of tanning using ultraviolet radiation, as occurs in the natural solar spectrum or is generated by artificial light sources, excessively long exposure or exceeding a certain dose may lead to not inconsiderable skin damage.

If the skin is exposed for too long to the sun or to a source of artificial rays, after a latency period of from 2 to 3 hours, a reddening of the skin, starkly demarcated from the unradiated skin, arises (solar erythema). Sunburn which has arisen in this way is differentiated as follows:

1st degree: erythema (reddening, feeling of warmth)—subsides again after 2 to 3 days and disappears with a simultaneous increase in pigmentation,

2nd degree: blister formation—blisters form on the skin with burning and itching, and sections of the epidermis are shed, and

3rd degree: cell damage—deep cell damage occurs, the body reacts with fever, large sections of the epidermis are shed.

The 2nd and 3rd degrees are also referred to as solar dermatitis.

The formation of the erythema is dependent on the wavelength. The erythema region of UV-B is between 280 nm and 320 nm.

Approximately 90% of the ultraviolet radiation which reaches the Earth consists of UV-A rays with a wavelength between 320 nm and 400 nm. Whereas UV-B radiation varies widely depending on numerous factors (e.g. season and time of day or latitude), UV-A radiation remains relatively constant from day to day irrespective of seasonal and diurnal or geographic factors. At the same time, most of the UV-A radiation penetrates into the living epidermis, while about 70% of the UV-B rays are retained by the horny layer.

For a long time, it has incorrectly been assumed that the long-wavelength UV-A radiation has only a negligible biological effect and that, correspondingly, the UV-B rays are responsible for most photodamage to the human skin. In the meantime, however, numerous studies have proven that UV-A radiation is far more hazardous than UV-B radiation with regard to the triggering of photodynamic, specifically phototoxic, reactions and chronic changes in the skin. The harmful effect of UV-B radiation can also be intensified by UV-A radiation.

Modern tanning facilities, as are used, for example, in solaria, produce predominantly UV-A radiation in a high irradiation intensity. Compared to the natural midday sun in summer, the UV-A irradiation intensity produced is generally higher by a factor of 3 to 5, and in individual cases even up to a factor of 10. At the same time, a small proportion of UV-B rays is emitted. The spectrum of rays is therefore generally more favorable with regard to tanning and less harmful than the natural solar spectrum with regard to the risk of sunburn.

The decisive advantage of solaria is the possibility of applying an exact and limited irradiation dose coupled with defined ray quality, which is hardly possible under natural conditions. However, particularly as a result of the increased UV-A irradiation intensity, a certain risk cannot of course be excluded.

Accordingly, anyone who wishes to avoid light-induced skin damage such as sunburn, chronic photodamage or a possible carcinoma risk, but on the other hand does not wish to sacrifice the fashionably desired tan must have recourse to those tanning methods which achieve results without the application of artificially generated or natural ultraviolet rays.

The simplest method of imparting a brown color shade to the skin is to apply appropriately colored make-up preparations. However, only those parts of the body which have been covered by the colored preparations are of course colored. A disadvantage of make-up is therefore the time-consuming application procedure. It is also disadvantageous that they rub off to a considerable extent onto textiles such as shirt collars or blouses. Moreover, the various dyes can have varying allergenic potential and even have a skin-irritative effect.

A further possibility of artificial tanning is to apply substances to the skin which undergo a chemical color reaction with the horny layer, resulting in an artificial tanning of the uppermost layers of the skin. The active ingredient which is most important and continues to be used most often in self-tanning preparations (so-called “self-tanners”) is dihydroxyacetone (DHA), a trivalent sugar which is present in the human body. DHA reacts with the proteins and amino acids in the horny layer, the color shade being achieved via a polymerization. The resulting color shade and the intensity of the coloration are dependent on the type of amino compounds and are influenced, for example, also by the preparation and the thickness of the horny layer.

This type of tanning is a purely chemical reaction of the horny layer. Although it achieves a certain cosmetic result, the tan peels off again very rapidly and is without any protective function for the skin. This tanning is completely independent of the melanocytes in the skin which bring about the natural skin tanning as a result of the pigment melanin formed by them.

Coloration by self-tanners takes place without the effect of sunlight. In contrast to this, so-called pretan products or tan promoters are also supplied; these have to be applied prior to solar irradiation. Then, in the sun, these preparations turn yellow, which is supposed to lead to a slight brown-yellow coloration of the epidermis, which additionally enhances the “sun tan”.

A further type of artificial tanning, which is likewise completely independent of UV light, can be caused by the hormones which are usually released in the body also as a result of (natural) UV irradiation and ultimately stimulate the melanocytes to synthesize melanin. In this connection, mention may be made, for example, of modifications of proopiomelanocortin (POMC), such as aMSH and synthetic variants (such as NDP), some of which have a much higher activity than the natural aMSH. Although these hormones in principle are able to bring about tanning, their use in cosmetics is prohibited since they are pharmacologically active substances (hormones), which should not be used widely without medicinal indication.

### SUMMARY OF THE INVENTION

An object of the present invention was thus to avoid the disadvantages of the prior art and to find substances for tanning the skin, as a result of the application of which a tanned appearance is imparted to the skin without the action of solar rays, and which intensify the existing natural tanning of the skin and prolong the natural skin tan.

In particular, these substances should be acceptable as regards health, be non-irritative and easy-to-handle, and the resulting color shade should correspond to the natural healthy skin color. The tanning achieved should be lightfast and not be removed by washing.

A further object of the present invention was to find cosmetic or dermatological preparations for tanning the skin.

It was surprising, and herein lies the achievement of these objects, that the disadvantages of the prior art are overcome by the use of one or more substances chosen from the group of AGEs and/or precursors thereof for tanning the skin.

### DETAILED DESCRIPTION OF THE INVENTION

The designation AGE stands for Advanced Glycosylation Endproducts. For the purposes of the present invention, AGEs are obtainable, for example, in accordance with the following (exemplary) reaction equation:

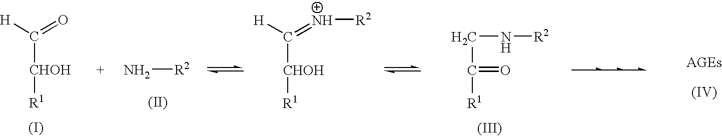

Advantageous for the purposes of the present invention are, as well as the actual AGEs, also their precursors, such as, for example, Amadori or Maillard products (corresponding to (III) in the above reaction equation) or so-called EGEs (Early Glycosylation Endproducts), which are obtainable, for example, as intermediates in the rearrangement of the Amadori products (III) to give the AGEs (IV).

Advantageous starting materials (I) carry an aldehyde group (CHO) and are chosen, for example, from the group of sugars (aldoses, e.g. trioses, tetroses, pentoses, hexoses and the like, such as glucose, galactose, mannose, etc.).

Advantageous starting materials (II) have a free amino group and are chosen, for example, from the group of amino acids and the group of peptides with terminal amino groups, in particular lysine, hydroxylysine, arginine, tryptophan and histidine are advantageous. The free amino group can also advantageously originate from N-terminal ends of proteins and peptides, additionally also advantageously sphingosine and dihydrosphingosine and homologs thereof with (unsaturated) acyl radicals of varying length.

Preferably, AGEs according to the invention contain at least one nitrogen-containing five- and/or six-membered ring.

Advantageous for the purposes of the present invention is, for example, so-called FFI, which is characterized by the following structural formula:

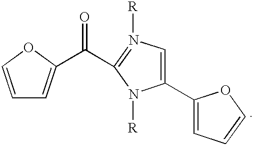

Also advantageous for the purposes of the present invention is so-called AFGP, which is characterized by the following structural formula:

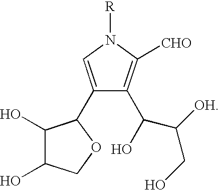

Also advantageous for the purposes of the present invention are pyrralines, which are characterized by the following structural formula:

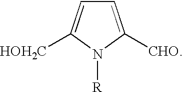

Advantageously according to the invention, the radical R originates, for example, from proteins and peptides; accordingly, it is here preferably a peptide, amino acid or protein radical, which may be joined to the nitrogen atom at the end or on the side.

R is also advantageously chosen, for example, from the group of saturated or unsaturated, branched or unbranched alkyl groups having 1 to 35 carbon atoms.

Also advantageous for the purposes of the present invention is pentosidine, which is characterized by the following structural formula:

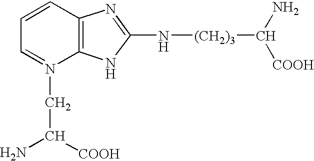

Particularly advantageous for the purposes of the present invention is also so-called A2E, which is characterized by the following structural formula:

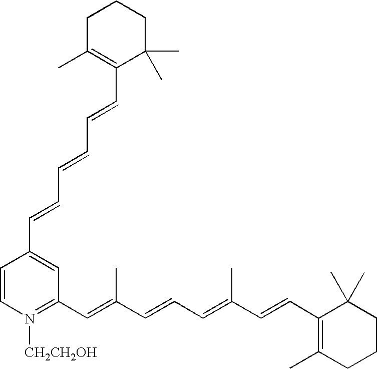

The structural formulae shown are of course not intended to limit the invention to certain isomers of the substances according to the invention. Rather, isomers which are not depicted and/or isomer mixtures are also advantageous for the purposes of the present invention.

The tanning substances for the purposes of the present invention are characterized, inter alia, by the fact that, following topical application, in the skin they induce the formation of pigments intrinsic to the skin, increase the synthesis of melanin and in this way produce an enhanced tanning of the skin. They are acceptable in terms of health, non-irritative and easy to handle, and the resulting color shade naturally corresponds to that of the natural healthy skin color. The resulting tan, since it corresponds to the natural tan, is lightfast and cannot be washed off. Surprising, the substances according to the invention also enhance the tanning of skin which is already tanned and, moreover, delay tanned skin from becoming pale.

A further advantage of the present invention arises from the protective properties of natural melanin formed in the skin. As well as various other functions of the melanin intrinsic to the skin (such as, for example, “detoxification” or binding of toxic substances and/or pharmaceuticals etc.), these functions of melanin are also in particular very important for the skin, inter alia with regard to homeostasis, the prevention of skin aging and the like. Melanin acts as a natural UV filter for protection against harmful UV rays, and moreover as an antioxidant for protecting against reactive oxygen species (oxidative stress), which can arise, inter alia, as a result of solar irradiation.

Thus, the use according to the invention (i.e. following topical application of the active ingredient according to the invention) results not only in a cosmetic benefit in the sense of enhanced tanning as a result of the increased melanine synthesis in the skin, but also an additional benefit as a result of the various protective powers of melanin.

Accordingly, the substances according to the invention are suitable in a particularly excellent manner as tanning active ingredients in cosmetic or dermatological preparations.

The present invention thus also provides cosmetic or dermatological formulations, characterized in that they comprise at least one substance chosen from the group of AGEs and/or precursors thereof.

The substances according to the invention and cosmetic or dermatological preparations comprising these induce, in the skin, the formation of pigments intrinsic to the skin, intensify the existing natural and/or artificial tanning of the skin and prolong the skin tan.

The formulations according to the invention are entirely satisfactory preparations in every respect which are characterized by a uniformly coloring action. The person skilled in the art could not have foreseen that the formulations according to the invention would

be easier to formulate,

more rapidly and better impart a naturally tanned appearance to the skin,

prolong skin tan,

have a better effect as moisturizing preparations,

better promote skin smoothing,

be characterized by better care action,

have better sensory properties, such as, for example, spreadability on the skin or the ability to be absorbed into the skin, and

offer a better/risk-free intrinsic protection of the skin (against UV radiation) than the preparations of the prior art. In addition, the formulations according to the invention, surprisingly, do not display any hormone effects.

Cosmetic or dermatological formulations for the purposes of the present invention can preferably, in addition to one or more oil phases, additionally comprise one or more water phases and be present, for example, in the form of W/O, O/W, W/O/W or O/W/O emulsions. Such emulsions may preferably also be a microemulsion, a Pickering emulsion or a sprayable emulsion.

Moreover, the formulations according to the invention can, however, also advantageously be in the form of oil-free preparations, such as, for example, gels, or in the form of anhydrous preparations.

It is advantageous to choose the content of one or more substances from the group of AGEs and/or precursors thereof in the cosmetic or dermatological formulations to be between 0.0001 and 30% by weight, particular advantageously between 0.01 and 10% by weight, very particularly between 0.1 and 2% by weight, in each case based on the total weight of the formulation.

The formulations according to the invention can further advantageously also comprise dihydroxyacetone or nut extracts, and also further substances which are said to retain or produce tanning.

The formulations according to the invention can advantageously, although not obligatorily, also be used in combination with UV radiation, be it with artificially produced or natural ultraviolet rays, for example in order to further increase the natural tanning or else in order to achieve a particularly long-lasting tan. For this purpose, the preparations according to the invention can advantageously, for example, be applied before, after or during the action of UV rays on the skin, for example in the form of a presun formulation, a sunscreen formulation or an aftersun formulation.

The cosmetic and dermatological and formulations according to the invention can have the customary composition and be used, in particular, for the treatment and care of the skin and/or the hair, as a make-up product in decorative cosmetics or as a sunscreen preparation or so-called presun or aftersun preparation. Accordingly, the formulations according to the invention can, depending on their formulation, be used, for example, as skin protection cream, face cream, cleansing milk, sunscreen lotion, nutrient cream, or day or night cream, etc.

For use, the cosmetic and dermatological formulations according to the invention are applied to the skin and/or the hair in a sufficient amount in the manner customary for cosmetics.

The cosmetic and dermatological formulations according to the invention may comprise cosmetic auxiliaries such as those customarily used in such preparations, e.g., preservatives, bactericides, perfumes, antifoams, dyes, pigments which have a coloring effect, thickeners, moisturizers and/or humectants, fats, oils, waxes or other customary constituents of a cosmetic or dermatological formulation, such as alcohols, polyols, polymers, foam stabilizers, electrolytes, organic solvents and/or silicone derivatives, and moisturizers.

Moisturizers is the term used to describe substances or mixtures of substances which, following application or distribution on the surface of the skin, impart to cosmetic or dermatological preparations the property of reducing the moisture loss by the horny layer (also called transepidermal water loss (TEWL)) and/or positively influencing hydration of the horny layer.

Advantageous moisturizers for the purposes of the present invention are, for example, glycerol, lactic acid, pyrrolidonecarboxylic acid and urea. It is also particularly advantageous to use polymeric moisturizers from the group of polysaccharides which are soluble in water and/or swellable in water and/or gelable using water. Particularly advantageous are, for example, hyaluronic acid and/or a fucose-rich polysaccharide which is listed in Chemical Abstracts under the registry number 178463-23-5 and is available, for example, under the name Fucogel®1000 from SOLABIA S.A.

The amounts of cosmetic or dermatological auxiliaries and carriers and perfume to be used in each case can easily be determined by the person skilled in the art by simple trial and error depending on the type of product in question.

An additional content of antioxidants is generally preferred. According to the invention, favorable antioxidants which can be used are any antioxidants suitable or conventional for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of gum benzoin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients, which are suitable according to the invention.

It is also advantageous if the cosmetic or dermatological formulations for the purposes of the present invention comprise enzymes or enzyme mimics which act as enzymatic antioxidants, such as, for example, superoxide dismutases, catalases, peroxidases, selenoproteins.

The amount of abovementioned antioxidants (one or more compounds) in the emulsions is preferably 0.001 to 30% by weight, particularly preferably 0.05 to 20% by weight, in particular 0.1 to 10% by weight, based on the total weight of the preparation.

If vitamin E and/or derivatives thereof are the antioxidant or antioxidants, it is advantageous to choose the respective concentrations thereof from the range from 0.001 to 10% by weight, based on the total weight of the formulation.

If vitamin A or vitamin A derivatives or carotenes or derivatives thereof are the antioxidant or the antioxidants, it is advantageous to choose the respective concentrations thereof from the range from 0.001 to 10% by weight, based on the total weight of the formulation.

The lipid phase can advantageously be chosen from the following group of substances:

mineral oils, mineral waxes

oils, such as triglycerides of capric or of caprylic acid, and also natural oils, such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates; and

silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously chosen from the group of esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, and from the group of esters of aromatic carboxylic acids and saturated and/or unsaturated, branched and/or unbranched alcohols with a chain length of from 3 to 30 C atoms. Such ester oils can then be advantageously chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic and natural mixtures of such esters such as, for example, jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and of fatty acid triglycerides, namely the triglycerol esters of saturated and/or unsaturated, branched and/or unbranched alkanecarboxylic acids having a chain length of from 8 to 24, in particular 12 to 18, carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, for the purposes of the present invention, paraffin oil, squalane and squalene are to be used advantageously.

The oil phase can also advantageously have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also to be used advantageously for the purposes of the present invention, for example, hexamethylcyclotrisiloxane, polydimethylsiloxane, poly(methylphenyl-siloxane).

Also particularly advantageous are mixtures of cyclomethicone and isotridecyl isononanoate, and of cyclomethicone and 2-ethylhexyl isostearate.

The aqueous phase of the formulations according to the invention may advantageously comprise alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can be chosen advantageously from the group consisting of silicon dioxide, aluminum silicates, polysaccharides and derivatives thereof, e.g. hyaluronic acid, xanthan gum and/or hydroxypropylmethylcellulose, particularly advantageously from the group consisting of polyacrylates, preferably a polyacrylate from the group of so-called “Carbopols”, e.g. Carbopol grades 980, 981, 1382, 2984, 5984, in each case individually or in combination.

Also favorable are those cosmetic and dermatological preparations which are in the form of a sunscreen. In addition to the active ingredients according to the invention, these preferably additionally comprise at least one UV-A filter substance and/or at least one UV-B filter substance. Such formulations may comprise, but do not necessarily comprise, optionally also one or more inorganic pigments as UV filter substances.

Preference is given to inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides.

An additional content of stabilizing titanium dioxide and/or zinc oxide particles may also of course be advantageous, but is not necessary for the purposes of the present invention.

It is also advantageous for the purposes of the present invention to provide those cosmetic and dermatological preparations whose main purpose is not protection against sunlight, but which nevertheless have a content of UV protection substances. Thus, for example, UV-A and/or UV-B filter substances are often incorporated into day creams.

UV protection substances, like antioxidants and, if desired, preservatives, also represent effective protection of the preparations themselves against decay.

Preparations according to the invention advantageously comprise substances which absorb UV radiation in the UV-A and UV-B region, the total amount of filter substances being, for example, from 0.1% by weight to 30% by weight, preferably from 0.5 to 20% by weight, in particular from 1.0 to 15.0% by weight, based on the total weight of the preparations, in order to provide cosmetic preparations which protect the hair or the skin from the whole range of ultraviolet radiation. They can also be used as sunscreens for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances are phenylene-1,4-bis(2-benzimidazyl)-3,3′-5,5′-tetrasulfonic acid, and its salts, particularly the corresponding sodium, potassium or triethanolammonium salts, in particular phenylene-1,4-bis(2-benzimidazyl)-3,3′-5,5′-tetrasulfonic bis-sodium salt, and 1,4-di(2-oxo-10-sulfo-3-bornylidenemethyl)benzene and salts thereof (in particular the corresponding 10-sulfato compounds, in particular the corresponding sodium, potassium or triethanolammonium salt), which is also referred to as benzene-1,4-di(2-oxo-3-bornylidenemethyl-10-sulfonic acid).

Advantageous UV filter substances for the purposes of the present invention are also broad-band filters, i.e. filter substances which absorb both UV-A and UV-B radiation.

Advantageous broad-band filters and/or UV-B filter substances are, for example, bisresorcinyltriazine derivates. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH, and to tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCl: Octyl Triazone), which is marketed by BASF Aktiengesellschaft under the trade name UVINUL® T 150.

A particularly preferred UV filter substance for the purposes of the present invention is also an asymmetrically substituted s-triazine, which is also referred to as dioctylbutylamidotriazone (INCI) and is available under the trade name UVASORB HEB from Sigma 3V.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl )-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1 -methylpyrrol-2-yl )-1,3,5-triazine, 2,4-bis{[4-tris(trimethylsiloxysilylpropyloxy)-2-hydroxy]phenyl}-6-(4-methoxyphenyl )-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,heptamethylsiloxy-2″-methylpropyloxy)-2-hydroxy]phenyl}-6-(4-methoxyphenyl)-1,3,5-triazine.

An advantageous broad-band filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

Another advantageous broad-band filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) having the INCl name Drometrizole Trisiloxane.

The UV-B filters can be oil-soluble or water-soluble. Examples of advantageous oil-soluble UV-B filter substances are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivates of benzophenone, preferably 2-hydroxy-4-methoxybenzophenone, 2-hydroxy-4-methoxy-4′-methylbenzophenone, 2,2′-dihydroxy-4-methoxybenzophenone, and

and UV filters bonded to polymers.

Examples of advantageous water-soluble UV-B filter substances are:

salts of 2-phenylbenzimidazole-5-sulfonic acid, such as its sodium, potassium or its triethanolammonium salt, and also the sulfonic acid itself; and

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance which can be used advantageously according to the invention is ethylhexyl 2-cyano-3,3-diphenylacrylate (octocrylene), which is available from BASF under the name Uvinul® N 539.

It can also be of considerable advantage to use polymer-bonded or polymeric UV filter substances in the preparations according to the present invention, in particular those described in WO-A-92/20690.

In some instances, it can also be advantageous to incorporate further UV-A and/or UV-B filters in accordance with the invention into cosmetic or dermatological preparations, for example certain salicylic acid derivatives, such as 4-isopropylbenzyl salicylate, 2-ethylhexyl salicylate (=octyl salicylate), homomenthyl salicylate.

The list of given UV filters which can be used for the purposes of the present invention is not of course intended to be limiting.

The examples below serve to illustrate the present invention without limiting it. Unless stated otherwise, all quantitative amounts, proportions and percentages are based on the weight and the total amount or on the total weight of the preparations.

### EXAMPLE 1

W/O cream

| | % by weight |
|---|---|
| Paraffin oil (German Pharmacopoeia (DAB) 9) | 10.00 |
| Petrolatum | 4.00 |
| Wool wax alcohol | 1.00 |
| PEG-7 hydrogenated castor oil | 3.00 |
| Aluminum stearate | 0.40 |
| Glycerol | 2.00 |
| Preservatives, dyes, perfume | q.s. |
| AFGP | 0.20 |
| Water | ad |
| | 100.00 |

### EXAMPLE 2

W/O lotion

| | % by weight |
|---|---|
| Paraffin oil (DAB 9) | 20.00 |
| Petrolatum | 4.00 |
| Glucose sesquiisostearate | 2.00 |
| Aluminum stearate | 0.40 |
| Phytoene | 1.00 |
| Glycerol | 5.00 |
| Preservatives, dyes, perfume | q.s. |
| Pentosidine | 2.50 |
| Water | ad |
| | 100.00 |

### EXAMPLE 3

O/W lotion

| | % by weight |
|---|---|
| Paraffin oil (DAB 9) | 8.00 |
| Isopropyl palmitate | 3.00 |
| Petrolatum | 4.00 |
| Cetylstearyl alcohol | 2.00 |
| PEG 40 castor oil | 0.50 |
| Sodium cetylstearyl sulfate | 0.50 |
| Sodium carbomer | 0.40 |
| Glycerol | 3.00 |
| α-Glucosylrutin | 0.20 |
| Octyl methoxycinnamite | 5.00 |
| Butylmethoxydibenzoylmethane | 1.00 |
| Preservatives, dyes, perfume | q.s. |
| AGE | 0.02 |
| Water | ad |
| | 100.00 |

### EXAMPLE 4

O/W cream

| | % by weight |
|---|---|
| Paraffin oil (DAB 9) | 7.00 |
| Avocado oil | 4.00 |
| Glyceryl monostearate | 2.00 |
| Sodium lactate | 3.00 |
| Glycerol | 3.00 |
| Preservatives, dyes, perfume | q.s. |
| FFI | 0.4 |
| Water | ad |
| | 100.00 |

### EXAMPLE 5

Liposome-containing gel

| | % by weight |
|---|---|
| Lecithin | 6.00 |
| Shea butter | 3.00 |
| Pentosidine | 0.20 |
| Sodium citrate | 0.50 |
| Glycine | 0.20 |
| Urea | 0.20 |
| Sodium PCA | 0.50 |
| Hydrolyzed collagen | 2.00 |
| Xanthan gum | 1.40 |
| Sorbitol | 3.00 |
| Preservatives, dyes, perfume | q.s. |
| A2E | 1.20 |
| Water | ad |
| | 100.00 |

### EXAMPLE 6

Sunscreen emulsion

| | % by weight |
|---|---|
| Cyclomethicone | 2.00 |
| Cetyldimethicone copolyol | 0.20 |
| PEG 22 dodecyl copolymer | 3.00 |
| Paraffin oil (DAB 9) | 2.00 |
| Caprylic/capric triglyceride | 5.80 |
| Octyl methoxycinnamate | 5.80 |
| Butylmethoxydibenzoylmethane | 4.00 |
| AGE | 0.50 |
| D-biotin | 0.50 |
| ZnSO₄ | 0.70 |
| Na₄EDTA | 0.30 |
| Preservatives, dyes, perfume | q.s. |
| Water | ad |
| | 100.00 |

### EXAMPLE 7

Sunscreen emulsion

| | % by weight |
|---|---|
| Cyclomethicone | 2.00 |
| Cetylstearyl alcohol + PEG 40 hydrogenated castor | 2.50 |
| oil + sodium | |
| cetylstearyl sulfate | |
| Glyceryl lanolate | 1.00 |
| Caprylic/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropylmethylcellulose | 0.30 |
| Octyl methoxycinnamate | 5.00 |
| Butylmethoxydibenzoylmethane | 0.50 |
| A2E | 0.20 |
| α-Tocopheryl acetate | 1.00 |
| Na₃HEDTA | 1.50 |
| Preservatives, dyes, perfume | q.s. |
| Water | ad |
| | 100.00 |

### EXAMPLE 8

Sunscreen emulsion

| | % by weight |
|---|---|
| Cyclomethicones | 2.00 |
| Cetylstearyl alcohol + PEG 40 hydrogenated castor | 2.50 |
| oil + sodium | |
| cetylstearyl sulfate | |
| Glyceryl lanolate | 1.00 |
| Caprylic/capric triglyceride | 0.10 |
| Laurylmethicone copolyol | 2.00 |
| Octyl stearate | 3.00 |
| Castor oil | 4.00 |
| Glycerol | 3.00 |
| Acrylamide/sodium acrylate copolymer | 0.30 |
| Hydroxypropylmethylcellulose | 0.30 |
| Octyl methoxycinnamate | 5.00 |
| Butylmethoxydibenzoylmethane | 0.75 |
| Na₃HEDTA | 1.50 |
| Preservatives, dyes, perfume | q.s. |
| A2E | 3.00 |
| Water | ad |
| | 100.00 |

### EXAMPLE 9

Spray formulation

| | % by weight |
|---|---|
| Ubiquinone 10 | 0.10 |
| Pyrralines | 0.80 |
| Ethanol | 28.20 |
| Preservatives, dyes, perfume | q.s. |
| Propane/Butane 25/75 | ad |
| | 100.00 |

### EXAMPLE 10

O/W cream

| | % by weight |
|---|---|
| Paraffin oil (DAB 9) | 7.00 |
| Soybean oil | 4.00 |
| Glyceryl monostearate | 2.00 |
| Sodium lactate | 3.00 |
| Glycerol | 8.00 |
| Preservatives, dyes, perfume | q.s. |
| A2E | 0.08 |
| Water | ad |
| | 100.00 |

That which is claimed:

1. A cosmetic or dermatological formulation, comprising at least one substance selected from the group consisting of AGEs and precursors thereof.

2. The formulation as claimed in claim 1, wherein the AGEs and precursors thereof are selected from the group consisting of FFI, pyrralines, pentosidine and A2E.

3. The formulation as claimed in claim 1, wherein the AGEs and precursors thereof are selected from the group consisting of AGEs.

4. The formulation as claimed in claim 3, wherein the AGEs include at least one nitrogen-containing five or six membered ring.

5. The formulation as claimed in claim 1, wherein the AGEs and precursors thereof are selected from the group consisting of EGEs.

6. The formulation as claimed in claim 5, wherein the EGEs are Amadori or Maillard products.

7. The formulation as claimed in claim 1, wherein the AGEs and precursors thereof are present in an amount between 0.0001 and 30% by weight, based on the total weight of the formulation.

8. The formulation as claimed in claim 1, wherein the AGEs and precursors thereof are present in an amount between 0.01 and 10% by weight, based on the total weight of the formulation.

9. The formulation as claimed in claim 1, wherein the AGEs and precursors thereof are present in an amount between 0.1 and 2% by weight, based on the total weight of the formulation.

10. The formulation as claimed in claim 1, said formulation in the form of an emulsion selected from the group consisting of W/O, O/W, W/O/W and O/W/O emulsions.

11. The formulation as claimed in claim 1, said formulation in the form of an emulsion selected from the group consisting of multiple emulsions, microemulsions, Pickering emulsions and sprayable emulsions.

12. The formulation as claimed in claim 1, said formulation in a form selected from the group consisting of presun formulations, sunscreen formulations and aftersun formulations.

13. A method comprising the step of applying to the skin a cosmetic or dermatological formulation comprising one or more substances selected from the group consisting of AGEs and precursors thereof.

14. The method as claimed in claim 13, wherein said cosmetic or dermatological formulation is applied to the skin for one or more of tanning the skin, increasing melanin synthesis, enhancing an existing tan, prolonging the brown coloration of the skin and protecting the skin against oxidative stress.

15. The method as claimed in claim 13, wherein the AGEs and precursors thereof are selected from the group consisting of FFI, pyrralines, pentosidine and A2E.

16. The method as claimed in claim 13, wherein the AGEs and precursors thereof are selected from the group consisting of AGEs.

17. The method as claimed in claim 16, wherein the AGEs include at least one nitrogen-containing five or six membered ring.

18. The method as claimed in claim 13, wherein the AGEs and precursors thereof are selected from the group consisting of EGEs.

19. The method as claimed in claim 18, wherein the EGEs are Amadori or Maillard products.

20. The method as claimed in claim 13, wherein the AGEs and precursors thereof are present in an amount between 0.0001 and 30% by weight, based on the total weight of the formulation.

21. The method as claimed in claim 13, wherein the AGEs and precursors thereof are present in an amount between 0.01 and 10% by weight, based on the total weight of the formulation.

22. The method as claimed in claim 13, wherein the AGEs and precursors thereof are present in an amount between 0.1 and 2% by weight, based on the total weight of the formulation.

23. A method for tanning the skin by applying to the skin one or more substances selected from the group consisting of AGEs and precursors thereof.

24. The method as claimed in claim 23, wherein the AGEs and precursors thereof are selected from the group consisting of FFI, pyrralines, pentosidine and A2E.

25. A method for increasing the melanin synthesis in the skin by applying to the skin one or more substances selected from the group consisting of AGEs and precursors thereof.

26. The method as claimed in claim 25, wherein the AGEs and precursors thereof are selected from the group consisting of FFI, pyrralines, pentosidine and A2E.

27. A method for enhancing the existing tan of the skin by applying to the skin one or more substances selected from the group consisting of AGEs and precursors thereof.

28. The method as claimed in claim 27, wherein the AGEs and precursors thereof are selected from the group consisting of FFI, pyrralines, pentosidine and A2E.

29. A method for prolonging the brown coloration of the skin by applying to the skin one or more substances selected from the group consisting of AGEs and precursors thereof.

30. The method as claimed in claim 29, wherein the AGEs and precursors thereof are selected from the group consisting of FFI, pyrralines, pentosidine and A2E.

31. A method for protecting the skin against oxidative stress by applying to the skin one or more substances selected from the group consisting of AGEs and precursors thereof.

32. The method as claimed in claim 31, wherein the AGEs and precursors thereof are selected from the group consisting of FFI, pyrralines, pentosidine and A2E.
